(19) United States
(12) Patent Application Publication
(10) Pub. No.: US 2005/0123509 A1
(43) Pub. Date: Jun. 9, 2005

(54) Modulating charge density to produce improvements in the characteristics of spray-dried proteins

(76) Inventors: S. Russ Lehrman, Los Altos, CA (US); Bing Yang, Redwood City, CA (US); Cynthia Stevenson, Mountain View, CA (US)

Correspondence Address: NEKTAR THERAPEUTICS, 150 INDUSTRIAL ROAD, SAN CARLOS, CA, 94070, US

(21) Appl. No.: 10/493,181

(22) PCT Filed: Oct. 16, 2002
(86) PCT No.: PCT/US02/33016

(30) Foreign Application Priority Data
Oct. 19, 2001 (US) 60330073

(51) Int. Cl. A61K 38/28; B29B 9/00; A61K 31/727; A61K 38/21; A61K 39/395

(52) U.S. Cl. 424/85.2; 514/12; 514/56; 424/130.1; 264/5; 424/85.4; 514/3

(57) ABSTRACT

Methods are provided for preparing spray-dried, drug-containing particles comprising the steps of selecting (i) a drug and an optional excipient, wherein the combination of the drug and optional excipient has an effective pI, and (ii) an aqueous solution having a pH that is different from the effective pI; (b) combining the solution and the drug and optional excipient, wherein an absolute net charge is associated with the drug and optional excipient as a result of an absolute difference between the pH and effective pI; and (c) spray drying the solution to form the spray-dried, drug-containing particles. Particles and compositions comprising the prepared particles as well as methods of use are also provided.

### FIELD OF THE INVENTION

The present invention relates generally to methods for preparing spray-dried, drug-containing particles, and more specifically to methods for improving, maintaining or optimizing the dispersibility of such particles. In addition, the invention relates to spray-dried, drug-containing particles, formulations comprising such particles, and methods for treating patients using the spray-dried, drug-containing particles.

### BACKGROUND OF THE INVENTION

Pulmonary delivery of therapeutic proteins is an effective route of administration that offers several advantages over conventional routes of administration. These advantages include, for example, the convenience of patient self-administration, the potential for reduced drug side-effects, the ease of delivery, the elimination of needles, and the like. Many preclinical and clinical studies with inhaled proteins, peptides, DNA and small molecules have demonstrated the efficacy of targeting local, i.e., within the lungs, and systemic delivery of therapeutic proteins.

Despite these initially encouraging results, however, the role of inhalation therapy in the health care field has not grown as expected over recent years, in part due to a set of problems unique to the development of inhalable drug formulations. In particular, dry powder formulations for pulmonary delivery, while offering unique advantages over liquid dosage forms and propellant-driven formulations, are often prone to agglomeration and low flowability phenomena. These and other phenomena considerably diminish the efficiency of delivery and the efficacy of dry powder-based inhalation therapies.

Spray drying is one of several well-known techniques for preparing dry powders. Other techniques include lyophilization, air-drying, spray freeze drying (as described in, for example, U.S. Pat. No. 6,284,282), and co-precipitation spray drying techniques, all of which have been used to prepare micron-sized powders. See, for example, WO 96/32149. Other methods for forming particles based on supercritical fluid technology are also known. See, for example, U.S. Pat. No. 6,063,138. Each technique, however, can produce particles that exhibit unsatisfactory properties such as agglomeration, low flowability, and so forth.

For example, spray drying has been employed with the aim of producing particles suitable for pulmonary inhalation. Spray drying techniques utilize a hot gas stream to evaporate microdispersed droplets created by atomization of a liquid feedstock to form dry powders. While spray drying has been long employed in the food and pharmaceutical industries to prepare dry powders, its application to therapeutic proteins has been rather limited because of the concern that certain proteins may be thermally degraded during the spray drying process. Although there is now a growing body of evidence to support the general utility of spray drying macromolecule-based biotherapeutic formulations to produce biologically active powders suitable for inhalation (as evidenced in WO 98/16205, WO 97/41833, WO 96/32152, WO 96/32116, WO 95/24183, and WO 01/00312), many peptides and proteins, when spray dried, form powders having limited dispersibilities. Due to their poor delivery profiles, powders having limited dispersibilities are unattractive for dry powder inhalation therapy.

Several aspects of the process of particle formation, especially during spray drying, can result in the production of particles that do not disperse well when emitted from an inhalation device. Problems have been associated with formulation components, spray-drying conditions, powder handling and packaging, and the like. Thus, the spray-drying process can result in the formation of dried particles that adhere to one another, i.e., agglomeration. As a result of increased size and other factors, agglomerated particles do not exit the inhalation device to the extent and in manner suitable for delivering a desired dose to a patient's lungs.

The present invention is therefore directed to methods for preventing or attenuating the agglomeration of drug-containing particles.

### SUMMARY OF THE INVENTION

Accordingly, it is a primary object of this invention to provide a method for preparing spray-dried, drug-containing particles comprising the steps of: (a) selecting (i) a drug and optional excipient, wherein the combination of the drug and optional excipient has an effective pI, and (ii) an aqueous solution having a pH that is different from the effective pI; (b) combining the solution and the drug and optional excipient, wherein an absolute net charge is associated with the drug and optional excipient as a result of an absolute difference between the pH and the effective pI; and (c) spray drying the solution to form spray-dried, drug containing particles.

It is another object of the invention to provide such a method further comprising the step of increasing the absolute net charge by increasing the difference between the pH and the effective pI.

It is an additional object of the invention to provide such a method wherein increasing the absolute net charge is effected by adding an acid to the solution when the pH is lower than the effective pI.

It is a still another object of the invention to provide such a method wherein increasing the absolute net charge is effected by adding a base to the solution when the pH is greater than the effective pI.

It is yet still another object of the invention to provide such a method wherein increasing the absolute net charge is effected by including the optional excipient in the solution, wherein the optional excipient serves as a charge-increasing excipient capable of increasing the absolute difference between the pH and effective pI.

It is an additional object of the invention to provide such a method wherein the charge-increasing excipient is selected from the group consisting of amino acids, oligopeptides, derivatives thereof, and combinations thereof.

It is still another object of the invention to provide such a method wherein the drug is a therapeutic protein.

It is a further object of the invention to provide a method for treating a patient comprising administering, via inhalation, the particles described herein.

Additional objects, advantages and novel features of the invention will be set forth in the description that follows, and in part, will become apparent to those skilled in the art upon the following, or may be learned by practice of the invention.

As a representation of hydrogen ion concentration, pH values provide information concerning the acidity or alkalinity of a solution. The pI or isoelectric point of a molecule is that pH at which the molecule has no net charge. As used herein, the effective pI represents the pH at which the overall positive and negative charges contributed from all of the components in the solution (i.e., the drug and any optional excipients) cancel each other out, thereby rendering the components (in totality) without a net charge. Thus, for example, in cases where the solution comprises a single drug, e.g., a single therapeutic protein without any excipients (i.e., a neat solution), the effective pI of the solution is the pI of the drug. In the case where one or more excipients are present in the solution, the effective pI is the weighted combination of the pI contributed from the drug and the excipient(s). The effective pI of a combination of a single drug and single excipient in solution can be estimated when the concentration of the excipient is much greater (e.g., 10-100 fold). In such a case, the effective pI is substantially identical to the pI of the excipient.

Thus, when the pH of the solution and the effective pI of the components contained therein are not the same, an absolute net charge associated with the components results. Preferably, the absolute difference (i.e., the distance from the pH to the effective pI and mathematically represented as |difference|) is at least about 0.2, more preferably at least about 0.5, still more preferably at least about 1.5, still more preferably at least about 2.5, still more preferably at least about 3.5, still more preferably at least about 4.5, with absolute differences of at least about 5.0 being most preferred.

The method can be optimized to increase the absolute difference between the pH and the effective pI. This can be accomplished in a number of different ways including, for example, by adding an acid to the solution when the pH is lower than the effective pI, adding a base to the solution when the pH is greater than the pI, and including the optional excipient in the solution, wherein the optional excipient serves as a charge-increasing excipient capable of increasing the absolute difference between the pH and effective pI. As will be further explained below, nonlimiting examples of charge-increasing excipients include those selected from the group consisting of amino acids, oligopeptides, derivatives thereof, and combinations thereof. Of course, any approach of increasing, the absolute difference between the pH and pI (e.g., by adding an acid or base) must not alter the drug's stability or solubility to the extent that a complete loss of therapeutic activity results. Those skilled in the art will recognize which approach or approaches are acceptable based upon routine experimentation and/or upon a reading of the description herein.

The particles prepared according the inventive method exhibit advantageous properties. For example, in another embodiment of the invention the particles can be included as part of a pharmaceutical formulation suitable for pulmonary delivery to a patient. The pharmaceutical formulation comprises the particles and an optional excipient. Preferably, the pharmaceutical formulation maintains its dispersibility over a twelve-week period. Maintenance of dispersibility, for example, can mean that the formulation exhibits a drop in emitted dose of no more than 25% over a twelve-week period. Advantageously, the particles prepared herein have a mass median aerodynamic diameter (MMAD) in the range between 0.1 μm to 5 μm. In addition, the density of the formulation is preferably in the range between 0.1 g/cm³ to 2 g/cm³.

In another embodiment of the invention, a method of treating a patient is provided based on administering to the patient a formulation comprising the spray-dried, drug-containing particles. The formulations can consist only of the spray-dried, drug-containing particles, or can comprise the spray-dried, drug-containing particles combined with one or more excipients. In this embodiment, a patient suffering from a condition that is responsive to drug therapy is administered, via inhalation, a therapeutically effective amount of the formulation described herein.

### DETAILED DESCRIPTION OF THE INVENTION

Before describing the present invention in detail, it is to be understood that this invention is not limited to the particular solution components, spray-drying techniques, drugs, and the like as such may vary. It is also to be understood that the terminology used herein is for describing particular embodiments only, and is not intended to be limiting.

It must be noted that, as used in this specification and the intended claims, the singular forms “a,” “an,” and “the” include plural referents unless the context clearly dictates otherwise. Thus, for example, reference to “a drug” includes a single drug as well as two or more different drugs, reference to a “an optional excipient” refers to a single optional excipient as well as two or more different optional excipients, and the like.

In describing and claiming the present invention, the following terminology will be used in accordance with the definitions described below.

The term “amino acid” refers to any molecule containing both an amino group and a carboxylic acid group. Although the amino group most commonly occurs at the position adjacent to the carboxy function, the amino group may be positioned at any location within the molecule. The amino acid may also contain additional functional groups, such as amino, thio, carboxyl, carboxamide, imidazole, and so forth. As used herein, the term “amino acid” specifically includes amino acids as well as derivatives thereof such as, without limitation, norvaline, 2-aminoheptanoic acid, and norleucine. The amino acid may be synthetic or naturally occurring, and may be used in either its racemic or optically active (D-, or L-) forms, including various ratios of stereoisomers. The amino acid can be any combination of such compounds. Most preferred are the naturally occurring amino acids. The naturally occurring amino acids (along with their common abbreviations) are: phenylalanine (phe or P); leucine (leu or L); isoleucine (ile or I); methionine (met or M); valine (val or V); serine (ser or S); proline (pro or P); threonine (thr or T); alanine (ala or A); tyrosine (tyr or Y); histidine (his or H); glutamine (gln or Q); asparagine (asn or N); lysine (lys or K); aspartic acid (asp or D); glutamic acid (glu or E); cysteine (cys or C); tryptophan (trp or W); arginine (arg or R); and glycine (gly or G).

As used herein, “therapeutic protein” is any polymer in which the monomers are amino acids, wherein the polymer has physiological activity upon administration to a patient. Often, but not necessarily, amide bonds link one amino acid monomer to another along the sequence. A “therapeutic protein” may include stereoisomers (e.g., D-amino acids) of the twenty conventional amino acids, unnatural amino acids, and other derivatives known to those skilled in the art. The therapeutic proteins used herein include natural and synthetically or recombinantly derived proteins, as well as analogs thereof, to the extent that they retain at least some degree of physiologic activity.

By “oligopeptide” is meant any polymer in which the monomers are amino acids totaling generally less than about 100 amino acids, preferably less than 25 amino acids. The term oligopeptide also encompasses polymers composed of two amino acids joined by a single amide bond as well as polymers composed of three amino acids.

An “aqueous solvent” refers to water or a mixed solvent system comprising water and one or more water-miscible co-solvents. “Aqueous solution” refers to a solution based on such a solvent, particularly to the solution from which the dried particles of the invention are formed. This solution may also be referred to as a “feed solution.”

“Dry” when used referring to a powder (e.g., as in “dry powder”) is defined as containing less than about 10% moisture. Preferred compositions contain less than 7% moisture, more preferably less than 5% moisture, even more preferably less than 3% moisture, and most preferably less than 2% moisture. The moisture of any given composition can be determined by the Karl Fischer titrimetric technique using a Mitsubishi moisture meter Model #CA-06. Moisture content may also be determined by thermal gravimetric analysis (TGA).

An “inhalable” dry powder that is “suitable for pulmonary delivery” refers to a composition comprising solid particles that is capable of (i) being readily dispersed in or by an inhalation device and (ii) inhaled by a subject so that at least a portion of the particles reach the lungs to permit penetration into the alveoli. Such a powder is considered to be “respirable” or “inhalable.”

“Aerosolized” particles are particles which, when dispensed into a gas stream by either a passive or an active inhalation device, remain suspended in the gas for an amount of time sufficient for at least a portion of the particles to be inhaled by the patient, so that a portion of the inhaled particles reaches the lungs. The “emitted dose” or “ED” is a value indicative of a dry powder's degree of aerosolization in a gas stream.

“Emitted dose” or “ED” provides an indication of the delivery of a drug formulation from a suitable inhaler device after a firing or dispersion event. More specifically, for dry powder formulations, the ED is a measure of the percentage of powder which is drawn out of a unit dose package and which exits the mouthpiece of an inhaler device. The ED is defined as the ratio of the dose delivered by an inhaler device to the nominal dose (i.e., the mass of powder per unit dose placed into a suitable inhaler device prior to firing). The ED is an experimentally determined parameter, and is typically established using an in vitro device set up to mimic patient dosing. To determine an ED value, a nominal dose of dry powder, typically in unit dose form, is placed into a suitable dry powder inhaler (such as that described in U.S. Pat. No. 5,785,049), which is then actuated, dispersing the powder. The resulting aerosol cloud is then drawn by vacuum from the device, where it is captured on a tared filter attached to the device mouthpiece. The amount of powder that reaches the filter constitutes the emitted dose. For example, for a 5 mg dry powder-containing dosage form placed into an inhalation device, if dispersion of the powder results in the recovery of 4 mg of powder on a tared filter as described above, then the emitted dose for the dry powder composition is: 4 mg (delivered dose)/5 mg (nominal dose)×100=80%. For non-homogenous powders, ED values provide an indication of the delivery of drug from an inhaler device after firing rather than of dry powder, and are based on amount of drug rather than on total powder weight. Similarly for propellant-containing metered-dose inhalers, the ED corresponds to the percentage of drug that is drawn from a dosage form and which exits the mouthpiece of an inhaler device. Emitted dose is used as a measure of dispersibility.

A “dispersible” or “aerosolizable” powder is one having an ED value of at least about 30%, more preferably 40-50%, and even more preferably at least about 50-60% or greater. A powder having superior aerosolizability possesses an ED value of at least about 65% or greater.

“Mass median diameter” or “MMD” is a measure of mean particle size, since the powders of the invention are generally polydisperse (i.e., consisting of a range of particle sizes). MMD values as reported herein are determined by centrifugal sedimentation, although any number of commonly employed techniques can be used for measuring mean particle size (e.g., electron microscopy, light scattering, laser diffraction and so forth). Instruments suitable for measuring MMD include, for example, the Horiba CAPA-700 particle size analyzer (Horiba Instruments Inc., Irvine, Calif.).

“Mass median aerodynamic diameter” or “MMD” is a measure of the aerodynamic size of a dispersed particle. The aerodynamic diameter is used to describe an aerosolized powder in terms of its settling behavior, and is the diameter of a unit density sphere having the same settling velocity, in air, as the particle. The aerodynamic diameter encompasses particle shape, density and physical size of a particle. As used herein, MMD refers to the midpoint or median of the aerodynamic particle size distribution of an aerosolized powder determined by cascade impaction, unless otherwise indicated. As known to those skilled in the art, an Andersen cascade Impactor (a sieve-like apparatus with a series of stages that capture particles on plates by inertial impaction according to their size, available from Thermo Anderson, Smyrna, Ga.) or other device can be used to determine MMD.

“Pharmacologically acceptable salt” includes, but is not limited to, salts prepared with inorganic acids, such as chloride, sulfate, phosphate, diphosphate, bromide, and nitrate salts, or salts prepared with an organic acid, such as malate, maleate, fumarate, tartrate, succinate, ethylsuccinate, citrate, acetate, lactate, methanesulfonate, benzoate, ascorbate, paratoluenesulfonate, palmoate, salicylate and stearate, as well as estolate, gluceptate and lactobionate salts. Similarly, salts containing pharmacologically acceptable cations include, but are not limited to, lithium, sodium, potassium, barium, calcium, aluminum, and ammonium (including alkyl substituted ammonium).

As used herein, an “excipient” is a nondrug component of a formulation. The excipient can be included in a solution that is subsequently spray dried and/or added to spray-dried particles. Furthermore, in the pulmonary delivery context, an excipient is one that can be taken into the lungs with no significant adverse toxicological effects to the patient.

“Pharmacologically effective amount” or “therapeutically effective amount” is the amount of drug needed to provide a desired therapeutic effect. The exact amount required will vary from subject to subject and will otherwise be influenced by a number of factors, as will be explained in further detail below. An appropriate “effective amount,” however, in any individual case can be determined by one of ordinary skill in the art using only routine experimentation.

As used herein, “pH” is defined as the negative logarithm (base 10) of the hydrogen ion concentration of a solution.

“pK” is a measurement of the degree of completeness of a reversible reaction, defined as the negative logarithm (base 10) of the equilibrium constant K; used, for example, to describe the extent of disassociation of a weak acid.

“pI” is the isoelectric point of a molecule, or the pH at which positive and negative charges on the molecule are balanced.

“Effective pI” is the term used to describe the dominant pI of a solution when several species with different pI's are present. Effective pI can be calculated as follows. For proteins, amino acids and poly amino acids, the charge (+or −) is calculated based on pKa of terminals and of functional side chains. Useful equations are shown below:

Any individual negative charge (for example: —COO—):

$$negative charge = \frac{Ka}{H + Ka} .$$

Any individual positive charge (for example: —NH₃⁺):

$$positive charge = \frac{H}{H + Ka} .$$

Total negative charge (n):

$$total negative charge = {\sum }_{l = 1}^{n} \frac{{Ka}_{l}}{H + {Ka}_{l}} .$$

Total positive charge (m):

$$total positive charge = {\sum }_{l = 1}^{m} \frac{H}{H + {Ka}_{l}} .$$

In general, “ambient conditions” are those in which the temperature is between 25° C. and the relative humidity is 60%.

The term “patient,” refers to a living organism suffering from or prone to a condition that can be prevented or treated by administration of a drug, and includes both humans an animals.

“Optional” or “optionally” means that the subsequently described circumstance may or may not occur, so that the description includes instances where the circumstance occurs and instances where it does not.

Turning to a first embodiment of the invention, the invention is directed to a method for preparing spray-dried, drug-containing particles comprising a step of selecting a drug and an optional excipient as well as an aqueous solution. The selecting step is carried out with the knowledge that the combination of the drug and optional excipient will have an effective pI, the pH of the solution will have pH, and the pH must be different from the effective pI. Another step in the method includes combining the solution and the drug and optional excipient, wherein an absolute net charge is associated with the drug and optional excipient as a result of an absolute difference between the pH and effective pI. Another step in the method comprises spray drying the solution to form the spray-dried, drug-containing particles. The invention is premised, in part, on the ability of many drugs such as therapeutic proteins to be charged based on the pH of the surrounding environment.

With respect to a therapeutic protein, depending on its primary sequence, the protein may contain one or more carboxylic acid, amine, guanidine, imidazole, or other functional groups. Moreover, because of these different functional groups, the net charge (or lack thereof) for any given protein will change depending on the pH of the surrounding environment. Therefore, changing the pH of the protein's environment will modulate the net charge of the protein contained therein. In the context of spray drying, the spray-dried solution results in dry particles comprising like charges at or near the surface. Without wishing to be bound by theory, it is believed that the repulsion forces associated with particles comprising like charges at their surfaces reduces particle aggregation, thereby resulting in particles having excellent aerosol characteristics. Although focusing on therapeutic proteins, one of ordinary skill in the art will appreciate the applicability of charge modulation to drugs possessing similar functional groups, e.g., carboxylic acid, amine, guanidine, imidazole, and so forth.

Any difference between the pH and effective pI results in an absolute net charge of the drug and optional excipient. Preferred absolute differences between the pH and the effective pI include those that are at least about 0.2, 0.5, 1.5, 2.5, 3.5, 4.5, and 5.0. It is understood that the absolute difference between the pH and effective pI is always represented as a positive value.

Advantageously, the present method for preparing spray-dried, drug-containing particles can include the further step of changing the absolute difference between the pH and the effective pI. Depending on the additional components used and the desires of the formulator, the absolute difference can be decreased or increased. Preferably, although not necessarily, the absolute difference between the pH and the effective pI is increased since such an increase generally (although not necessarily) results in an increase in the absolute net charge of the component(s) in solution. An increase in the absolute net charge of the component(s) in solution generally results in a decrease in particle aggregation upon spray drying of the solution.

Any method for increasing the absolute difference between the pH and the effective pI can be used and the invention is not limited in this respect. For example, the absolute net charge associated with the solution components can be effected by changing the pH of the solution. Specifically, adding an acid to the solution when the pH is lower than the effective pI will increase the absolute net charge. In addition, when the pH is greater than the effective pI, adding a base can increase the absolute net charge. Furthermore, the absolute net charge can be increased by ensuring that the optional excipient is not only present in the solution, but also serves as a charge-increasing excipient capable of increasing the absolute difference between the pH and effective pI. Of course, combinations of approaches may also be used such as adding both an acid and a charge-increasing excipient. Each of these approaches is discussed in further detail below.

When adding an acid to the solution in order to increase the absolute difference between the pH and the effective pI (with the aim of increasing the absolute net charge), the pH must be lower than the effective pI. The relationship can be represented as shown below: 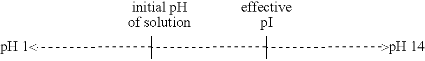
Thus, the addition of an appropriate acid will lower the pH of the solution, thereby increasing the absolute difference between the pH and effective pI. In this case, components (e.g., drug and any excipients) having a pI higher than the pH will have a net positive charge.

The added acid, however, should result in a more acidic solution. Such acids for any given solution can be determined by those of ordinary skill in the art or may be determined through routine methods. For example, an appropriate acid can be identified by measuring the pH of the solution (by, e.g., using a conventional pH meter) before and following the addition of the proposed acid. In the present context, an appropriate acid is one that will, upon addition to the solution, decrease the pH of the solution. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof.

In addition, the addition of a base can increase the absolute difference between the pH and the effective pI when the pH is greater than the effective pI. The relationship can be represented as shown below: 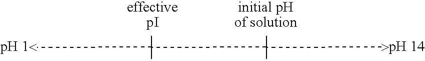
Here, addition of an appropriate base will increase the pH of the solution as well as the absolute difference between the pH and effective pI. In this case, components (e.g., drug and any excipients) having a pI lower than the pH will have a net negative charge.

For maximum effectiveness, the addition of the base must result in a more basic solution. Again, such bases can be determined by those of ordinary skill in the art or may be determined through routine methods such as measuring the pH before and after the addition of the proposed base. Examples of suitable bases include, without limitation, bases selected from the group sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Moreover, the absolute net charge can be increased by ensuring that the optional excipient is not only present but also capable of increasing the absolute difference between the pH and effective pI. In this approach, the effective pI is moved away from the pH of the solution through the addition of a charge-increasing excipient.

Any excipient capable of increasing the absolute difference between the pH and effective pI can be used and the invention is not limited in this regard. Preferred charge-increasing excipients include amino acids, oligopeptides, derivatives thereof, and combinations thereof.

Exemplary amino acids that act as charge-increasing excipients include those selected from the group consisting of glycine, alanine, valine, norvaline (2-aminopentanoic acid), 2-aminoheptanoic acid, leucine, isoleucine, methionine, proline, phenylalanine, tryptophan, serine, threonine, cysteine, tyrosine, asparagine, glutamic acid, lysine, arginine, histidine, norleucine, and combinations thereof. Preferred amino acids include those selected from the group consisting of leucine, isoleucine, norleucine, valine, norvaline, 2-aminoheptanoic acid, phenylalanine, tryptophan, and combinations thereof. It is particularly preferred, however, to use amino acids selected from the group consisting of leucine, isoleucine, norleucine, and combinations thereof as charge-increasing excipients.

Oligopeptides for use as a charge-increasing excipient comprise 2-9 amino acids, more preferably 2-5 amino acids. Although dipeptides (comprising two amino acid residues) and tripeptides (comprising three amino acid residues) can include any amino acid or derivative thereof, those dipeptides and tripeptides that include the amino acids (either one or more) selected from the group consisting of leucine, isoleucine, valine, norleucine, phenylalanine, and tryptophan are particularly preferred. Dipeptides and tripeptides containing two or more leucine residues can also be used as charge-increasing excipients and are also preferred. See, for example, WO 01/32144. In particular, dileucine and trileucine are preferred charge-increasing excipients. A dileucine-containing tripeptide can contain two leucine residues at any position, i.e., adjacent to each other or one at each terminus of the tripeptide, with the remaining position being occupied by any other amino acid. Preferably, however, the remaining amino acid residue in dileucine-containing tripeptide is selected from the group consisting of leucine, valine, isoleucine, tryptophan, alanine, methionine, phenylalanine, tyrosine, histidine, and proline. Preferred examples of oligopeptidies include those selected from the group consisting of dileucine leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, and combinations thereof.

For oligopeptides comprising four or five amino acids, these oligopeptides preferably comprise two or more leucine residues occupying any position. Preferably, although not necessarily, the nonleucine amino acids in an oligopeptide comprising four or five amino acids are hydrophilic in nature (e.g., such as lysine), thereby increase the solubility of the peptide in water.

In addition, notwithstanding any of the specifically mentioned charge-increasing excipients, those charge-increasing excipients that can increase the glass transition temperature (Tg) can render the drug-containing formulation more stable. It has been found that adding an excipient with a relatively high glass transition temperature can increase the glass transition temperature of the overall formulation in which the excipient is found. Therefore, excipients having a glass transition temperature greater than about 40° C., more preferably greater than 50° C., even more preferably greater than 60° C., and most preferably greater than 70° C., are preferred.

Moreover, charge-increasing excipients will preferably have relatively low solubilities in water, e.g., from about 10 mg/ml to about 75 mg/ml. Although not bound by theory, reduced aqueous solubility may result in decreased moisture sorption and delayed crystallization in the resulting spray-dried particles, both of which are desirable characteristics for respirable particles.

Also preferred are charge-increasing excipients having relatively large Van der Waals volumes, e.g., greater than about 100 Å³. Exemplary charge-increasing excipients having relatively large Van der Waals volume include isoleucine, leucine, lysine, methionine and phenylalanine. An increase in Van der Waals volume has been correlated with an increase in the glass transition temperature of the resulting spray-dried particles, thus indicating greater storage stability. Hydrophobic charge-increasing excipients such as leucine, valine, isoleucine, tryptophan, alanine, methionine, phenylalanine, tyrosine, histidine, and proline are also preferred.

Another property preferred in charge-increasing excipients is the ability to decrease the surface tension of water, which correlates with lower MMDs and reduced protein aggregation in the resulting spray-dried particles. Specific examples of charge-increasing excipients that lower the surface tension of water include asparagine, isoleucine, phenylalanine, tryptophan, tyrosine, leucine, and valine.

The choice of the specific charge-increasing excipient will depend upon the desired contribution the charge-increasing excipient will make to the effective pI. Generally, however, the charge-increasing excipient is used to move the effective pI away from the pH of the solution. For systems in which the pH is lower than the pI without the excipient, the charge-increasing excipient is selected so as to increase the effective pI. A representation is provided below:

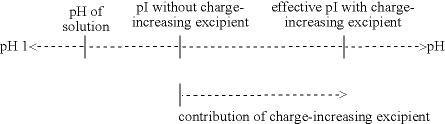

Similarly, when the pH is greater than the pI without the excipient, the charge-increasing excipient is selected so as to decrease the effective pI. A representation is provided below:

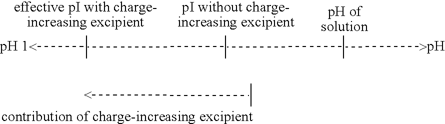

Thus, selecting the charge-increasing excipient becomes a matter of knowing the pH of the solution, the isoelectric point of the solution component(s), i.e., the drug, and the expected contribution of the charge-increasing excipient. As discussed above, the pH of the solution can easily be established through routine testing with a pH meter.

Isoelectric points for any component in the solution are known to those of ordinary skill in the art and/or can be obtained. For example, an isoelectric point can be determined experimentally by electrophoresing the protein over a pH gradient created in a polyacrylamide gel. By electrophoresing a mixture of polyampholytes having many pI values, gels with the necessary pH gradient can be created. Also, gels with the necessary pH gradient are commercially available such as those manufactured by Bio-Rad Laboratories (Hercules Calif.) under the name ReadyStrip. Once electrophoretic movement of the component ends, the component can be visualized on the gel via application of a suitable dye (e.g., Coomassie blue). The pH corresponding to the location where the component rests is equal to the pI of the protein. In addition, computer software such as the GCG® Wisconsin Package® software collection (available from Accelrys, San Diego Calif.) include programs for calculating the pI of proteins. As shown in Table 1, many drugs such as therapeutic proteins have isoelectric points between 5 and 9.5.

TABLE 1
Isoelectric Points (pI) for Various Drugs

| Drug | pI |
|---|---|
| Interferon beta (glycosylated) | 6.6-6.8 |
| Insulin | 6.90 |
| Human growth hormone | 5.37 |
| Calcitonin | 8.90 |
| Alpha-1 antitrypsin (human) | 5.4 |
| Parathyroid hormone | 9.10 |

The charge-increasing excipient's expected contribution to the effective pI can be determined by identifying the excipient's own pI with the understanding that greater amounts of the excipient bring the effective pI closer to the excipient's pI. The same procedures used above with respect to determining the pI for drugs can be used to determine the pI of the charge-increasing agent. In addition, the pKa's of any functional groups on the charge-increasing excipient is often a predictor of pI. The pKa's of functional groups for representative charge-increasing excipient are provided in Table 2.

TABLE 2
Functional Groups and pKa's of Representative Charge-Increasing
Excipients

| Charge-Increasing Excipient | Functional Group | pKa |
|---|---|---|
| Aspartic acid | β-carboxylic acid | 3.9 |
| Glutamic acid | γ-carboxylic acid | 4.3 |
| Histidine | δ1-N imidazole | 6.0 |
| Lysine | ε-amino | 10.5 |
| Arginine | δ-guanidino | 12.5 |

For each functional group listed in Table 2, roughly 90% will be in ionized or neutral forms at one pH unit away from its respective pKa value

Thus, once the three variables, i.e., the pH of the solution, the pI of the drug, and the effective pI, are known (or estimated), the absolute difference between the pH and the effective pI can be modulated to optimize the absolute net charge. Generally, such optimization involves increasing the absolute difference between pH and the effective pI. For example, by adding lysine with a relatively high pKa function group of 10.5 to a salmon calcitonin (pI 9.3) solution at pH 7, the absolute difference the between pH and effective pI is increased. The absolute net charge of other systems can also be modulated based on the same principles.

When increasing the absolute difference between the pH and effective pI, it is controlled so that the increase does not lead to deleterious chemical modification and, in the case of therapeutic proteins, irreversible denaturation. Such chemical modifications and/or irreversible denaturation can lead to a loss of the drug's activity. Routine experimentation such as administering the formulation to a patient and monitoring for the expected therapeutic response can be used to determine whether the drug has lost activity.

As stated above, the solution comprises a drug, typically a therapeutic protein. Suitable drugs for use in the present invention include, for example, erythropoietin (EPO), Factor VIII, Factor IX, prothrombin, thrombin, alpha-1 antitrypsin, alglucerase, imiglucerase, cyclosporin, granulocyte colony stimulating factor (GCSF), thrombopoietin (TPO), alpha-1 proteinase inhibitor, elcatonin, calcitonin, granulocyte macrophage colony stimulating factor (GMCSF), human growth hormone (hGH), growth hormone releasing hormone (GHRH), heparin, low molecular weight heparin (LMWH), interferon alpha, interferon beta, interferon gamma, interleukin-1 receptor, interleukin-2, interleukin-1, interleukin-1 receptor antagonist, interleukin-3, interleukin-4, interleukin-6, interleukin-7, interleukin-8, interleukin-9, interleukin-10, interleukin-11, interleukin-12, interleukin-13 receptor, luteinizing hormone releasing hormone (LHRH), leuprolide, nafarelin, goserelin, buserelin, insulin, pro-insulin, insulin analogues (e.g., mono-acylated insulin as described in U.S. Pat. No. 5,922,675), amylin, C-peptide, somatostatin, octreotride, vasopressin, follicle stimulating hormone (FSH), insulin-like growth factor (IGF), insulinotrophin, macrophage-colony stimulating factor (M-CSF), nerve growth factor (NGF), platelet-derived growth factor (PDGF), basic fibroblast growth factor (bFGF), acidic fibroblast growth factor (aFGF), stem cell factor (SCF), oncostatin M, heparin-derived growth factor (HGF), herceptin, epidermal growth factor (EGF), endothelial cell growth factor (ECGF), vascular growth factor (VGF), thyroxin, tissue growth factors, keratinocyte growth factor (KGF), glial growth factor (GGF), tumor necrosis factor (TNF), endothelial growth factor, parathyroid hormone (PTH), glucagon, thymosin alpha 1, IIb/IIIa inhibitor, phosphodiesterase (PDE) inhibitors, VLA-4 inhibitors, bisphosphonates, respiratory syncytial virus antibody, cystic fibrosis transmembrane regulator (CFTR) gene, deoxyribonuclease (Dnase), bactericidal/permeability increasing protein (BPI), anti-CMV antibody, any therapeutic monoclonal or polyclonal antibody, pharmacologically acceptable salts of any of the foregoing as well as combinations of any of the foregoing. Particularly suitable for use in the methods and compositions described herein are growth factor hormones, parathyroid hormone, leuprolide, calcitonin, insulin, interferon alpha, interferon beta, interferon gamma, follicle stimulating hormone, leutinizing hormone releasing hormone (LHRH), human growth hormone, pharmacologically acceptable salts thereof, and combinations of any of the foregoing. The therapeutic proteins can be naturally derived or synthesized using recombinant or chemical techniques known to those of ordinary skill in the art. In addition, several therapeutic proteins are available from commercial suppliers such as, for example, Sigma (St. Louis, Mo.).

The amount of the drug in the formulation administered to the patient will typically contain at least about one of the following percentages of active agent: 1%, 5%, 10%, 20%, 30%, 40%, 50%, 60%, 70%, 80%, 90% or more by weight. Preferably, the spray-dried powder will contain at least about 50%, e.g., from about 50 to 100% by weight of the drug. For particularly potent drugs, however, low concentrations can be used.

The solution can also contain or more other optional excipients, none of which necessarily serves as a charge-increasing excipient. Although the invention is not limited in this regard, such optional excipients preferably include those selected from the group consisting of carbohydrate excipients, inorganic salts, antimicrobial agents, antioxidants, surfactants, and combinations thereof.

Suitable for use in protecting the drug during spray drying are carbohydrate excipients such as sugars, derivatized sugars such as alditols, aldonic acids, esterified sugars, and sugar polymers. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. Preferred are non-reducing sugars, sugars that can form an amorphous or glassy phase with a drug in a spray-dried solid, and sugars possessing relatively high glass transitions temperatures or “Tgs” (e.g., Tgs greater than 40° C., preferably greater than 50° C., more preferably greater than 60° C., and even more preferably greater than 70° C., and most preferably having Tgs of 80° C. and above). Particularly preferred stabilizing excipients are sucrose, mannitol and trehalose.

The compositions may further include an inorganic salt such as sodium chloride, potassium chloride, sodium sulfate, potassium nitrate, and the like. Upon dissociation, salts provide ions, which further increase charge density, thereby decreasing aggregation. Salts that provide monovalent or divalent cations such as aluminum, manganese, calcium, zinc, and magnesium are preferred. When present, such cations are typically present in relative molar amounts ranging from about 50:1 (cation [mol]/drug [mol] to about 1:1, more preferably between about 20:1 to 2:1).

The solution may also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the solution as well. Antioxidants are used in the solution to prevent oxidation, thereby preventing the deterioration of the drug. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The solution may also include a surfactant in order to facilitate the spray-drying process. Exemplary surfactants include: polysorbates, such as “TWEEN 20” and “TWEEN 80,” and pluronics such as F68 and F88 (both of which are available from BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; and chelating agents, such as EDTA, zinc and other such suitable cations. One preferred excipient combination includes a pluronic (e.g., F68) and trileucine.

Protein excipients, which serve to increase the stability of the drug, may be present in the solution or formulation administered to the patient. Exemplary protein excipients include, without limitation, albumins such as human serum albumin (HSA), recombinant human albumin (rHA), gelatin, casein, hemoglobin, and the like. Such proteinaceous excipients, if employed, will contribute to the effective pI.

Preferably, although not necessarily, permeation enhancers (e.g., dimethylsulfoxide) and buffers are not present in the solution or final formulation administered to the patient.

Other optional excipients suitable for use in the compositions according to the invention are listed in “Remington: The Science & Practice of Pharmacy,” 19^{th} ed., Williams & Williams, (1995), “Physician's Desk Reference, 52^{nd} ed., Medical Economics, Montvale, N.J. (1998), WO 96/32096, and in “Handbook of Pharmaceutical Excipients,” 3^{rd} ed., Kibbe, A. H. Editor (2000).

The amount of any individual excipient (when present) in the solution or in the final formulation administered to the patient will vary depending on the activity of the excipient and particular needs of the formulation. Typically, the optimal amount of any individual excipient is determined through routine experimentation, i.e., by preparing compositions containing varying amounts of the excipient (ranging from low to high), examining protein aggregation, MMADs and dispersibilities of the resulting spray-dried compositions, and then further exploring the range at which optimal aerosol performance is attained with no significant adverse effects.

Generally, however, the excipient will be present in the solution or formulation administered to the patient in an amount of about 1% to about 99% by weight, preferably from about 5%-98% by weight, more preferably from about 15-95% by weight excipient, with concentrations less than 30% by weight most preferred.

Once the solution and the drug and optional excipient have been selected, the components are combined into the solution and mixed. The drug is first added to water or a similar aqueous system. Preferably, the drug is dissolved in an aqueous solution. The pH range of drug-containing solution is generally between about 3 and 7, more typically between about 3 to 5, and most preferably between about 3.5 to 4.

The solution can optionally contain water-miscible solvents, such as acetone, alcohols and the like. Representative alcohols suitable for this purpose include lower alcohols such as methanol, ethanol, propanol, and isopropanol. Such mixed solvent systems typically contain from about 0-80% of the water miscible solvent, more preferably from about 20-40%, and most preferably from about 10-30% of the water miscible solvent. The pre-spray dried solutions will generally contain solids dissolved at a concentration from 0.01% (weight/volume) to about 20% (weight/volume), usually from 0.05% to 10% (weight/volume), and preferably from about 0.1 to 2% (weight/volume). In particular, the pre-spray dried formulation will typically possess one of the following solids concentrations: 0.1 mg/ml or greater, 0.5 mg/ml or greater, 1 mg/ml or greater, 1.5 mg/ml or greater, 2 mg/ml or greater, 3 mg/ml or greater, 4 mg/ml or greater, or 5 mg/ml or greater. When the drug is a protein, the protein can be spray dried at a solids concentration of 0.1 mg/ml, which is effective to provide a spray-dried solid in which conformation of the native protein is preserved. Preferably, however, the maximum amount of solids content will be used when the drug is a therapeutic protein so that relatively high amounts of the protein are found in each droplet, thereby decreasing the potential for denaturing. It is believed that the likelihood of denaturing increases when the protein molecules have access to the air-liquid interface.

Once the components have been combined and any further steps of adjusting the pH and/or adjusting the effective pI have been carried out, the solution is spray dried according to conventional spray-drying techniques. Spray drying of the solution can be carried out, for example, as described in “Spray Drying Handbook,” 5^{th} ed., K. Masters, John Wiley & Sons, Inc., NY, N.Y. (1991), WO 97/41833 and WO 96/32149.

For example, the solutions can be spray dried in a conventional spray drier, such as those available from commercial suppliers such as Niro A/S (Denmark), Buchi (Switzerland) and the like, resulting in a dispersible, dry powder. Optimal conditions for spray drying the solutions will vary depending upon the solution components, and are generally determined experimentally. The gas used to spray dry the material is typically air, although inert gases such as nitrogen or argon are also suitable. Moreover, the temperature of both the inlet and outlet of the gas used to dry the sprayed material is such that it does not cause decomposition or degradation of the drug in the sprayed material. Such temperatures are typically determined experimentally, although generally, the inlet temperature will range from about 50° C. to about 200° C., while the outlet temperature will range from about 30° C. to about 150° C. Preferred parameters include atomization pressures ranging from about 20 to 150 psi (0.14 to 1.03 MPa), and preferably from about 30-40 to 100 psi (0.21-0.28 to 0.69 MPa). Typically the atomization pressure employed will be one of the following: 20 psi (0.14 MPa), 30 psi (0.21 MPa), 40 psi (0.28 MPa), 50 psi (0.34 MPa), 60 psi (0.41 MPa), 70 psi (0.48 MPa), 80 psi (0.55 MPa), 90 psi (0.62 MPa), 100 psi (0.69 MPa), 110 psi (0.76 MPa), 120 psi (0.83 MPa) or above.

The ability of the charged components, i.e., the drug and optional excipient, to retain the charge on drying results in repulsion between like-charged components, as well as between particles. These repulsive properties result in a decrease in aggregration, increase in dispersion and reduction of compaction from shipping and storage. In particular, it is surprising that activity of the drug can be maintained when, in order to provide an absolute net charge, the drug is placed in a relatively low pH environment. As is known to those of ordinary skill in the art, low pH environments can lead to degradation processes such as deamidation, asparagine rearrangement to isoaspartic acid, cleavage at Asp-Pro linkages, and dehydration of serine and threonine residues. See, for example, Lai et al. (1999) J. Pharm. Sci. 88(5):489-500 and Volkin et al. (1997) Mol. Biotechnol. 8(2):105-22. Therapeutic proteins are particularly prone to degradation through these processes. Moreover, many therapeutic proteins are known to partially lose tertiary structure and form a partially unfolded intermediate known as a “molten globule.” See, for example, Dodson (1994) Curr. Biol. 4(7):636-640. That the method for forming drug-containing, spray-dried particles does not result in a substantial loss of activity is unexpected.

The drug-containing, spray-dried particles will have a charge on their surface as a result of the absolute charge associated with the drug and optional excipient. In this respect, the greater the surface charge, the greater the degree of repulsion between particles and the greater the dispersibility. The total surface charge for any particle prepared according to the present method can be measured based on its zeta potential. Zeta potential measurement can be accomplished by using a commercially available dynamic light scattering instrumentation. The net charge is measured by monitoring the movement of a charged particle of a known size in response to an electric field.

Spray dried powders are physically distinct from powders prepared by other evaporative drying methods, and typically exhibit morphologies and thermal histories (including glass transition temperatures, glass transition widths, and enthalpic relaxation profiles) that differ from those of powders prepared by other drying methods such as lyophilization. Once formed, the protein dry powder compositions are preferably maintained under dry conditions (i.e., relatively low humidity). Irrespective of the particular drying parameters employed, the spray drying process results in inhalable, nonaggregated, highly dispersible particles comprising the drug.

The drug-containing, spray-dried particles can be administered “as is” or in combination with one or more optional excipients as discussed previously. In each case, the drug-containing, spray-dried particles are part of a powder formulation (either consisting exclusively of the particles or including one or more optional excipients). In all cases, the powder formulation is characterized by (i) consistently high dispersibilities, which are maintained, even upon storage (ii) small aerodynamic particles sizes (MMADs), (iii) improved fine particle dose values (e.g., powders having a higher percentage of particles sized less than 3.3 microns MMAD). These characteristics all advantageously contribute to the ability of the powder to penetrate into the lower respiratory tract (e.g., the alveoli). Once in the lower respiratory tract, the drug can act locally or systemically.

The drug-containing, spray-dried particles generally have a mass median diameter (MMD) of less than about 20 μm, preferably less than about 10 μm, more preferably less than about 7.5 μm, and still more preferably less than about 4 μm, with mass median diameters less than about 3.5 μm being most preferred. Expressed in a range, the drug-containing, spray-dried particles are preferably in the range of about 0.1 μm to 5 μm in diameter, preferably from about 0.2 to 4.0 μm. When an optional excipient is added to the drug-containing, spray-dried particles, the excipient can have the same size as the spray-dried particles, although the particle size of any excipient can also be larger and nonrespirable. With respect to the later, a carbohydrate carrier such as lactose serving as a carrier may have a particle size of about greater than 40 microns in size can be added to drug-containing, spray-dried particles produced in accordance with the invention.

The particles and powder formulations of the invention may further be characterized by density. The particles and powder formulations will generally possess a bulk density of from about 0.1 to 10 g/cm³, preferably from about 0.1 to 2 g/cm³, and more preferably from about 0.15 to 1.5 g/cm³.

The particles and powder formulations will generally have a moisture content below about 20% by weight, usually below about 10% by weight, and preferably below about 6% by weight. More preferably, the particles and powder formulations will typically possess a residual moisture content below about 3%, more preferably below about 2%, and most preferably between about 0.5 and 2% by weight. Such low moisture-containing solids tend to exhibit a greater stability upon packaging and storage. Generally, the particles of powder formulations of the invention are hygroscopic, i.e., moisture absorbing. Therefore, the particles and powder formulations can be stored in sealed containers such as blister packages to prevent hygroscopic growth.

An additional measure for characterizing the overall aerosol performance of particles and powder formulations is the fine particle fraction (FPF), which describes the percentage of powder having an aerodynamic diameter less than 3.3 microns. The particles and powder formulations are particularly well suited for pulmonary delivery, and possess FPF values ranging from about 30% to 64% or more. Preferred particles and formulation powders contain at least about 30 percent of aerosol particle sizes below 3.3 μm to about 0.5 μm and are thus extremely effective when delivered in aerosolized form.

The particles and powder formulations described herein also possess chemical and physical stability over time. Generally, with respect to chemical stability, the drug contained in the formulation will degrade by no more than about 10% upon spray drying. Stated differently, the drug-containing, spray-dried particles possess at least about 90% intact drug, preferably at least about 95% intact drug, and even more preferably will contain at least about 97% intact drug.

Preferably, the particles and powder formulations have less than about 10% total aggregates, preferably less than about 7% total aggregates, and most preferably less than 5% total aggregates. More specifically, the particles and powder formulations typically possess less than about 10% insoluble aggregates, preferably less than 7% insoluble aggregates, and most preferably less than 5% insoluble aggregates. Insoluble aggregates can be measured by ultraviolet spectroscopy (UV) using a Shimadzu UV-2101 PC dual spectrophotometer scanning over a range of 360 to 240 nm. In addition, insoluble aggregates can also be determined quantitatively by measuring the turbidity of the solution.

With respect to soluble aggregates, the particles and powder formulations typically contain less than 7% soluble aggregates, preferably less than 4% soluble aggregates, more preferably less than 2% soluble aggregates, and most preferably less than 1% soluble aggregates. For drugs that can form dimers or higher oligomers (e.g., therapeutic proteins), the total amount of monomer in the particles is typically greater than 90%, more preferably greater than 95%, and most preferably greater than 98%. Soluble aggregates can be determined by size exclusion high pressure liquid chromatography.

Moreover, the particles and powder formulations of the invention further demonstrate good stability upon storage. For example, when the drug is a therapeutic protein, the total protein aggregate content is less than 10% after storage for one month at 40° C. and ambient relative humidity. With respect to aerosol performance, the particles and powder formulations exhibit a drop in emitted dose of no more than about 20%, preferably no more than about 15%, and more preferably by no more than about 10%, when stored under ambient conditions for a period of three months.

The improvement in aerosol properties noted for the particles and powder formulations results in several related advantages, such as: (i) reducing costly drug losses to the inhalation device, since more powder is aerosolized and is therefore available for inhalation by a patient; (ii) reducing the amount administered, due to high aerosolization efficiency, and (iii) reducing the number of inhalations per day by increasing the amount of aerosolized drug that reaches the lungs of a patient.

The invention also provides a method for treating a patient suffering from a condition that is responsive to treatment with the drug. The method of treatment involves administering to the patient, via inhalation, formulations comprising the described particles (either alone or combined with one or more excipients added after the formation of the spray-dried particles). The method of treatment may be used to treat any condition that can be remedied or prevented by administration of the particular drug. Those of ordinary skill appreciate which conditions a specific drug can effectively treat. The actual dose to be administered will depend upon the age, weight, and general condition of the subject as well as the severity of the condition being treated, the judgment of the health care professional, and specific drug being used. Therapeutically effective amounts are known to those skilled in the art and/or are described in the pertinent reference texts and literature. Generally, an effective amount will range from about 0.001 mg/day to 100 mg/day, preferably in doses from 0.01 mg/day to 75 mg/day, and more preferably in doses from 0.10 mg/day to 50 mg/day.

The particles and powder formulations described herein may be administered using any suitable dry powder inhaler (DPI). Briefly, some DPIs utilize the patient's inhaled breath as a vehicle to transport the dry powder drug to the lungs. Generally, the powder is contained in a receptacle having a puncturable lid or other access surface, preferably a paper or foil surface of a blister package or cartridge, where the receptacle may contain a single dosage unit or multiple dosage units. Each dose may be weighed separately using a conventional scale. In addition, convenient methods are available for filling large numbers of cavities (i.e., unit dose packages) with metered doses of dry powder medicament. See, for example, WO 97/41031. For a description of various DPIs and how they work, reference is made to U.S. Pat. Nos. 5,458,135, 5,740,794, and 5,785,049, and WO 01/00263.

Other types of DPIs suitable for delivering the particles and powder formulations described herein include those that use a hard gelatin capsule containing a premeasured dose. See, for example, U.S. Pat. Nos. 3,906,950 and 4,013,075.

Other dry powder dispersion devices for pulmonary administration include those described in, for example, European Pat. Nos. EP 129985, EP 472598, and EP 467172 and U.S. Pat. No. 5,522,385. Also suitable is the TURBUHALER device available from Astra-Draco. This type of device is described in detail in U.S. Pat. Nos. 4,668,281, 4,667,668, and 4,805,811. Other suitable devices include dry powder inhalers such as the Rotahaler® (Glaxo), Discus® (Glaxo), Spiros™ (Dura Pharmaceuticals), and Spinhaler® (Fisons) inhalers. Also suitable are devices that use a piston to provide air for either entraining powdered medicament, lifting the medicament from a carrier screen by passing air through the screen, or mixing air with powder medicament in a mixing chamber with subsequent introduction of the medicament to the patient through the mouthpiece of the device. See, for example, U.S. Pat. No. 5,388,572.

The particles or powder formulations may also be delivered using a pressurized, metered dose inhaler (MDI). The particles or powder formulation are dissolved or suspended in a pharmaceutically inert liquid propellant, e.g., a chlorofluorocarbon, fluorocarbon or hydrogen-containing fluorocarbon. See, for example, U.S. Pat. Nos. 5,320,094 and 5,672,581. In addition, the particles and powder formulations described herein may be dissolved or suspended in a solvent, e.g., water, ethanol or saline, and administered by nebulization. Nebulizers for delivering an aerosolized solution include the AERx™ (Aradigm), the Ultravent® (Mallinkrodt), and the Acorn II® (Marquest Medical Products) devices.

Prior to use, the particles and/or powder formulations are generally stored under ambient conditions, and preferably are stored at temperatures at or below about 25° C., and relative humidities (RH) ranging from about 30 to 60%. More preferred relative humidity conditions, e.g., less than about 30%, can be achieved by incorporating a desiccating agent in the secondary packaging of the dosage form. Particles and powder formulations may also be stored under “accelerated” stability at 40° C., relative humidity 75%, for the purpose of determining stability.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All articles, books, patents and other publications referenced herein are hereby incorporated by reference in their entireties.

### Experimental

The practice of the invention will employ, unless otherwise indicated, conventional techniques of pharmaceutical formulating and the like, which are within the skill of the art. Such techniques are fully explained in the literature. See, for example, Remington, The Science and Practice of Pharmacy, supra.

In the following examples, efforts have been made to ensure accuracy with respect to numbers used (e.g., amounts, temperatures, etc.) but some experimental error and deviation should be accounted for. Unless indicated otherwise, temperature is in degrees C. and pressure is at or near atmospheric pressure at sea level. All reagents were obtained commercially unless otherwise indicated.

### EXAMPLE 1

A human growth hormone (hGH) formulation for pulmonary delivery was prepared. Methionyl-human growth hormone (Met-hGH, pI 5.2), obtained from BreSagen Limited (Adelaide, SA) was mixed at a concentration of 7 mg/mL (70% w/w) with trileucine (pI 5.9, Bachem Calif. Inc., Torrance, Calif.) at concentrations of 1.5 and 3 mg/mL, in separate solutions. Each solution was then divided and one solution was adjusted to a pH of 3.6 with an acid while the other was adjusted to a pH of 7.8 with a base. Individually, the solutions were spray dried to form particles using a Buchi 190 laboratory scale drier (Buchi, Switzerland) under the following conditions: feed rate: 5 ml/min; outlet temperature: 60° C.; and atomization pressure: 80 psi (0.55 MPa). The dispersibility of these Met-hGH particles are listed in Table 3 below:

TABLE 3
Emitted Dose (ED) Results for Spray-Dried Met-hGH Formulations
at Two pH and Two Trileucine Levels

| | 15% trilecuine | 30% trileucine |
|---|---|---|
| pH 3.6 | 92% ED | 88% ED |
| pH 7.8 | 75% ED | 73% ED |

In addition, particles produced from spray drying a formulation at pH 7.8 that lacked trileucine were found to have an ED of 76%.

The data show that the Met-hGH formulations at a lower pH provide enhanced dispersibility of the corresponding spray-dried powder. The relationships between pI and pH can shown as provided below: 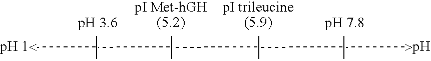
Without trileucine, the effective pI of the formulation is the 5.2, pI of hGH. Upon spray drying of the trileucine-free formulation at pH 7.8, an ED of 76% was measured. The addition of trileucine (at both 15% and 30% levels) with a pI of 5.9 increases the effective pI of the Met-hGH and trileucine combination. Now the effective pI must lie somewhere between 5.2 and 5.9, thereby bringing the effective pI closer to pH 7.8 and representing a decrease in the absolute difference between the pH and the effective pI. As expected, the ED values of spray-dried particles prepared from both trileucine-containing formulations at pH 7.8 decreased compared to particles prepared from the trileucine-free formulation. Because the absolute difference between pH and effective pI is less at pH 3.6 than at pH 7.8, there appears to be a preference for positively charged moieties, which are associated with low-pH formulations.

### EXAMPLE 2

An interferon beta formulation for pulmonary delivery was prepared. Interferon beta, obtained from Biogen, Inc. (Cambridge, Mass.), at a concentration of 1 mg/mL was mixed with raffinose at a concentration of 9 mg/mL and titrated to pH 4.0 with HCl. A pH of 4.0 lies 2 to 2.5 pH units below the pI of the fully glycosylated interferon beta. The solution was spray dried to form particles using a Buchi 190 laboratory scale drier (Buchi, Switzerland) under the following conditions: feed rate: 5 ml/min; outlet temperature: 65° C.; and atomization pressure: 100 psi (0.69 MPa). The ED for this formulation was 67% (±8%). The ED for formulations at a higher pH (e.g., pH 5) could not be determined due to the presence of protein aggregates.

### EXAMPLE 3

An interleukin-4 receptor formulation for pulmonary delivery was prepared. Soluble interleukin-4 receptor, obtained from Immunex, Inc. (Seattle, Wash.), was mixed with raffinose and citrate at pH 4 and 7. The mixture had a total solids content of 5 to 10 mg/mL. Excipient components represented 5 to 15% of the total solids content. Each solution was spray dried to form particles using a Buchi 190 laboratory scale drier (Buchi, Switzerland) under the following conditions: feed rate: 5 ml/min; outlet temperature: 70° C.; atomization pressure: 100 psi (0.69 MPa). The ED values of the pH 4.0 and 7.3 formulations were 71 and 66%, respectively.

### EXAMPLES 4-9

Following the general procedures set forth in Examples 1-3, six additional drug-containing formulations were evaluated. The drug, formulation, and pH for each formulation are provided in Table 4. In addition, Table 4 also shows the total charge densitity as well as the absolute net charge, when measured. The following abbreviations are used in the table: hGH for human growth hormone; sCT for salmon calcitonin; β-INF for interferon beta; PTH for parathyroid hormone; FSH for follicle stimulating hormone; SDS for sodium dodecyl sulfate; HES for hydroxyethylstarch; and standard three-letter abbreviations for all amino acids.

TABLE 4
Emitted Dose (ED) Results for Spray-Dried, Drug-Containing Formulations

| | | | | Total | Absolute | |
|---|---|---|---|---|---|---|
| | | | | Charge | Net | |
| Drug | pI | Formulation | pH | Density | Charge | ED |
| HGH | 5.3 | 100% hGH | 3.6 | 0.0013 | 0.0008 | 89 |
| | | 70% hGH, 30% trileucine | 3.6 | 0.0013 | 0.0008 | 90 |
| | | 70% hGH, 30% trileucine | 5.3 | 0.0022 | 0.0000 | 85 |
| | | 100% hGH | 7.8 | 0.0021 | 0.0003 | 71 |
| | | 70% hGH, 30% trileucine | 7.8 | 0.0021 | 0.0003 | 71 |
| | | 70% hGH, 30% leu | 7.8 | 0.0021 | 0.0003 | 74 |
| SCT | 9.3 | 100% sCT | 5.0 | 0.0019 | 0.0009 | 84 |
| | | 100% sCT | 7.0 | 0.0015 | 0.0006 | 77 |
| | | 100% sCT | 11.0 | 0.0013 | 0.0002 | 75 |
| | | 5% sCT, 10% mannitol, 65% citrate, 20% ala | 7.0 | 0.0140 | 0.0005 | 73 |
| | | 5% sCT, 10% mannitol, 65% citrate, 20% leu | 7.0 | 0.0140 | 0.0005 | 67 |
| | | 5% sCT, 10% mannitol, 65% citrate, 20% his | 7.0 | 0.0130 | 0.0000 | 63 |
| | | 5% sCT, 10% mannitol, 65% citrate, 20% lys | 7.0 | 0.0200 | 0.0060 | 71 |
| Insulin | 6.4 | 60% insulin, 10% mannitol, 2.6% gly, 2.3% Na | 7.3 | 0.0017 | 0.0004 | 80 |
| β-INF | 7.8 | 10% INF, 90% mannitol | 2.0 | | | 51 |
| | | 10% INF, 90% leu | 4.0 | 0.0130 | 0.0015 | 72 |
| | | 10% INF, 90% raffinose | 4.0 | | | 61 |
| | | 10% INF, 50% HES, 40% raffinose | 4.0 | | | 69 |
| | | 10% INF, 50% HES, 40% raffinose | 6.0 | | | 70 |
| | | 10% INF, 90% buffer, 1% SDS | 7.0 | | | 40 |
| TH | 9.0 | 30% PTH, 70% mannitol | 3.7 | | | 60 |
| | | 5% PTH, 10% mannitol, 65% citrate, 20% phe | 7.0 | 0.0140 | 0.0005 | 61 |
| | | 5% PTH, 10% mannitol, 65% citrate, 20% met | 7.0 | 0.0140 | 0.0005 | 65 |
| | | 5% PTH, 10% mannitol, 65% citrate, 20% ala | 7.0 | 0.0140 | 0.0005 | 60 |
| | | 5% PTH, 10% mannitol, 65% citrate, 20% val | 7.0 | 0.0140 | 0.0005 | 63 |
| | | 5% PTH, 10% mannitol, 65% citrate, 20% leu | 7.0 | 0.0140 | 0.0005 | 61 |
| | | 5% PTH, 10% mannitol, 65% citrate, 20% his | 7.0 | 0.0130 | 0.0005 | 41 |
| SH | 5.3 | 5% FSH, 95% mannitol | 7.0 | | | 50 |
| | | 5% FSH, 95% raffinose | 7.0 | | | 55 |
| | | 5% FSH, 15% mannitol, 80% citrate | 7.0 | | | 50 |

As indicated in Table 4, changing the pH and/or using charge-increasing excipients can advantageously increase the absolute net charge of the drug with the concomitant result of providing a formulation that, upon spray drying, exhibits an effective dose suited for pulmonary administration.

1. A method for preparing spray-dried, drug-containing particles comprising the steps of:
(a) selecting (i) a drug and an optional excipient, wherein the combination of the drug and optional excipient has an effective pI, and (ii) an aqueous solution having a pH that is different from the effective pI;
(b) combining the solution and the drug and optional excipient, wherein an absolute net charge is associated with the drug and optional excipient as a result of an absolute difference between the pH and effective pI; and
(c) spray drying the solution to form the spray-dried, drug-containing particles.

2. The method of claim 1, wherein the absolute difference between the pH and effective pI is at least about 0.2.

3. The method of claim 2, wherein the absolute difference between the pH and effective pI is at least about 0.5.

4. The method of claim 3, wherein the absolute difference between the pH and effective pI is at least about 1.5.

5. The method of claim 4, wherein the absolute difference between the pH and effective pI is at least about 2.5.

6. The method of claim 5, wherein the absolute difference between the pH and effective pI is at least about 3.5.

7. The method of claim 6, wherein the absolute difference between the pH and effective pI is at least about 4.5.

8. The method of claim 7, wherein the absolute difference between the pH and effective pI is at least about 5.0.

9. The method of claim 1, further comprising the step of increasing the absolute net charge by increasing the absolute difference between the pH and the effective pI.

10. The method of claim 9, wherein the step of increasing the absolute net charge is effected by adding an acid to the solution when the pH is lower than the effective pI.

11. The method of clam 10, wherein the acid is selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof.

12. The method of claim 9, wherein the step of increasing the absolute net charge is effected by adding a base to the solution when the pH is greater than the effective pI.

13. The method of claim 12, wherein the base is selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

14. The method of claim 9, wherein the step of increasing the absolute net charge is effected by including the optional excipient in the solution, wherein the optional excipient serves as a charge-increasing excipient capable of increasing the absolute difference between the pH and effective pI.

15. The method of claim 9, wherein the charge-increasing excipient is selected from the group consisting of amino acids, derivatives of amino acids, oligopeptides, derivatives thereof, and combinations thereof.

16. The method of claim 15, wherein the charge-increasing excipient is an amino acid or derivative thereof.

17. The method of claim 16, wherein the amino acid or derivative thereof is selected from the group consisting glycine, alanine, valine, norvaline, 2-aminoheptanoic acid, leucine, isoleucine, methionine, proline, phenylalanine, tryptophan, serine, threonine, cysteine, tyrosine, asparagine, glutamic acid, lysine, arginine, histidine, norleucine, and combinations thereof.

18. The method of claim 17, wherein the amino acid or derivative thereof is selected from the group consisting of leucine, isoleucine, norleucine, valine, norvaline, 2-aminoheptanoic acid, phenylalanine, tryptophan, and combinations thereof.

19. The method of claim 18, wherein the amino acid or derivative thereof is selected from the group consisting of leucine, isoleucine, norleucine, and combinations thereof.

20. The method of claim 15, wherein the charge-increasing excipient is an oligopeptide.

21. The method of claim 20, wherein the oliogopeptide is selected from the group consisting of dileucine, leu-leu-gly, leu-leu-ala, leu-leu-val, leu-leu-leu, leu-leu-ile, leu-leu-met, leu-leu-pro, leu-leu-phe, leu-leu-trp, leu-leu-ser, leu-leu-thr, leu-leu-cys, leu-leu-tyr, leu-leu-asp, leu-leu-glu, leu-leu-lys, leu-leu-arg, leu-leu-his, leu-leu-nor, leu-gly-leu, leu-ala-leu, leu-val-leu, leu-ile-leu, leu-met-leu, leu-pro-leu, leu-phe-leu, leu-trp-leu, leu-ser-leu, leu-thr-leu, leu-cys-leu, leu-try-leu, leu-asp-leu, leu-glu-leu, leu-lys-leu, leu-arg-leu, leu-his-leu, leu-nor-leu, and combinations thereof.

22. The method of claim 1, wherein the optional excipient is present in the solution.

23. The method of claim 22, wherein the excipient is selected from the group consisting of carbohydrate excipients, inorganic salts, antimicrobial agents, antioxidants, surfactants, and combinations thereof.

24. The method of claim 23, wherein the excipient is a carbohydrate excipient.

25. The method of claim 24, wherein the carbohydrate excipient is selected from the group consisting of fructose, maltose, galactose, glucose, mannose, sorbose, lactose, sucrose, trehalose, cellobiose, raffinose, melezitose, maltodextrans, dextrans, starches, mannitol, xylitol, lactitol, glucitol, pyranosyl sorbitol, myoinositol, and combinations thereof.

26. The method of claim 1, wherein the drug is a therapeutic protein.

27. The method of claim 26, wherein the therapeutic protein is selected from the group consisting of erythropoietin, Factor VIII, Factor IX, prothrombin, thrombin, alpha-1 antitrypsin, alglucerase, imiglucerase, cyclosporin, granulocyte colony stimulating factor, thrombopoietin, alpha-1 proteinase inhibitor, calcitonin, elcatonin, granulocyte macrophage colony stimulating factor, growth hormone, human growth hormone, growth hormone releasing hormone, heparin, low molecular weight heparin, interferon alpha, interferon beta, interferon gamma, interleukin-1 receptor, interleukin-2, interleukin-1, interleukin-1 receptor antagonist, interleukin-3, interleukin-4, interleukin-6, interleukin-7, interleukin-8, interleukin-9, interleukin-10, interleukin-11, interleukin-12, luteinizing hormone releasing hormone, leuprolide, goserelin, nafarelin, buserelin, insulin, pro-insulin, insulin analogues, amylin, C-peptide, somatostatin, octreotride, vasopressin, follicle stimulating hormone, insulin-like growth factor, insulinotrophin, macrophage colony stimulating factor, nerve growth factor, platelet derived growth factor, basic fibroblast growth factor, acidic fibroblast growth factor, stem cell factor, oncostatin M, heparin derived growth factor, herceptin, epidermal growth factor, endothelial cell growth factor, vascular growth factor, thyroxin, tissue growth factor, keratinocyte growth factor, glial growth factor, tumor necrosis factor, endothelial growth factors, parathyroid hormone, glucagon, thymosin alpha 1, IIb/IIIa inhibitor, phosphodiesterase inhibitors, VLA-4 inhibitors, bisphosphonates, respiratory syncytial virus antibody, cystic fibrosis transmembrane regulator gene, deoxyribonuclease, bactericidal/permeability increasing protein, therapeutic monoclonal antibodies, therapeutic polyclonal antibodies, pharmacologically acceptable salts thereof, and combinations thereof.

28. The method of claim 1, wherein the therapeutic protein is selected from the group consisting of such as parathyroid hormone, calcitonin, insulin, interferon, follicle stimulating hormone, luteining hormone releasing hormone, leuprolide, growth hormone, pharmacologically acceptable salts thereof, and combinations thereof.

29. Spray-dried, drug-containing particles prepared according to claim 1.

30. A pharmaceutical formulation comprising the spray-dried, drug-containing particles of claim 1 and an optional excipient.

31. The formulation of claim 30, wherein dispersibility of the formulation is maintained over a 12-week period.

32. The formulation of claim 31, wherein the formulation exhibits a drop in emitted dose of no more than 25% over a 12-week period.

33. The formulation of claim 30, wherein the moisture content of the formulation is less than 6% by weight.

34. The formulation of claim 30, wherein the formulation is suitable for inhalation.

35. The formulation of claim 30, wherein the MMAD of the spray-dried, drug-containing particles is in the range between 0.1 μm to 5 μm.

36. The formulation of claim 30, wherein the bulk density of the formulation is in the range between 0.1 g/cm³ to 2 g/cm³.

37. The formulation of claim 30, wherein the formulation contains the optional excipient.

38. The formulation of claim 37, wherein the optional excipient is selected from the group consisting of carbohydrate excipients, inorganic salts, antimicrobial agents, antioxidants, surfactants, and combinations thereof.

39. A method for treating a patient suffering from a condition that is responsive to treatment with a therapeutic drug comprising administering, via inhalation, a therapeutically effective amount of a pharmaceutical formulation of claim 30.
